(19) United States
(12) Patent Application Publication
(10) Pub. No.: US 2024/0285181 A1
(43) Pub. Date: Aug. 29, 2024

(54) DEVICE FOR POSITIONING A FETAL HEART RATE TRANSDUCER

(71) Applicant: COOBABYBOO LTD, Basildon Essex (GB)

(72) Inventor: Ethel SAKUTUKWA, Basildon Essex (GB)

(21) Appl. No.: 18/571,183

(22) PCT Filed: Jun. 15, 2022
(86) PCT No.: PCT/IB2022/055542

§ 371 Date: Dec. 15, 2023

(30) Foreign Application Priority Data
Jun. 15, 2021 (GB) 2108496.7

(51) Int. Cl. A61B 5/024; A61B 5/00

(52) U.S. Cl. CPC A61B 5/02411; A61B 5/02438; A61B 5/6831; A61B 5/6843

(57) ABSTRACT

A device for positioning a fetal heart rate transducer against a pregnant women's abdomen. The device includes a body having a lower surface and an upper surface opposite the lower surface. The lower surface is non-planar and includes a protruding portion intended to engage an upper surface of the transducer in use and configured such that the protruding portion can be positioned to apply a force to a selected area on the upper surface of the transducer.

### TECHNICAL FIELD

The present invention relates generally to devices for positioning a fetal heart rate transducer against an expectant mother's abdomen.

### BACKGROUND

External cardiotocography (CTG) is used during pregnancy to record fetal heart rate (FHR) and uterine contractions. The machine used to perform this monitoring, a cardiotocograph or electrical fetal monitor (EFM), uses an ultrasound transducer and a tocodynamometer transducer mounted on the expectant mother's abdomen. The ultrasound transducer is positioned in as optimal position as possible to monitor fetal heart rate and the tocodynamometer transducer at the fundus of the uterus to measure frequency of contractions.

CTG monitoring is especially important when caring for high-risk pregnant women, with most key decisions in planning the care of high-risk mothers and babies being guided with the help of CTG monitoring. Failure to monitor properly can lead to delays in identifying abnormalities leading to corresponding delays in necessary intervention.

One widespread problem encountered during CTG monitoring is the loss of a fetal heart rate (FHR) signal, which causes anxiety for the mother being monitored and their families and concern for medical personnel. Most commonly, loss of an FHR signal is due to a change in fetal position or a loss of contact between the FHR transducer and the mother's skin. Occasionally, however, the loss of an FHR signal can conceal signs of fetal distress that requires prompt attention. Thus, accurate monitoring is important.

To help hold the FHR transducer in position against the mother's abdomen, it is conventional to use one or more belts worn around the torso that can be used to secure the transducer in place.

However, these belts do not reliably hold the transducer in place, especially when the mother moves and, in many cases, medical personnel (e.g. a midwife) must regularly re-position the transducer to ensure a good FHR signal is maintained. This is time consuming, and, in some cases, a midwife may find it necessary to apply pressure to the transducer to hold it in a suitable position for 30 minutes or more to obtain an acceptable CTG recording. Clearly this is inefficient use of a midwife's time, and can be disconcerting for the pregnant mother, as well as potentially resulting in painful, numb fingers for the midwife and/or bad posture leading to back pain.

Where external CTG monitoring becomes difficult or unreliable, medical personnel may decide to use internal monitoring, typically involving the use of fetal scalp electrodes, which can be contraindicated in some pregnancies.

WO2019118564 describes a device for securing a FHR transducer in place using a strap secured to adhesive patches. U.S. Pat. No. 7,854,237 describes a disc shaped product with four chambers that can be inflated or deflated to adjust the pressure applied to a transducer.

### SUMMARY OF INVENTION

Embodiments of the invention are generally aimed at providing devices that can be used to more reliably hold an FHR transducer in position on a pregnant women's abdomen.

In a first aspect, the invention provides a device for positioning a fetal heart rate transducer against a pregnant women's abdomen, the device comprising:
a body having a lower surface and an upper surface opposite the lower surface;
wherein the lower surface is non-planar and comprises a protruding portion intended to engage an upper surface of the transducer in use and configured such that the protruding portion can be positioned to apply a force to a selected area on the upper surface of the transducer.

In use, the body of the device is located with the protruding portion of the lower surface engaged with the top surface of the transducer. By appropriately selecting the position on the top surface of the transducer against which the protruding portion engages, the angle at which the device applies pressure to the transducer can be adjusted to obtain a good FHR signal. This mimics the way in which a midwife might use the palm of their hand to apply pressure to the transducer.

In some cases, the weight of the body of the device may be sufficient by itself to apply the required pressure. If needs be, however, the device can be held in place with a belt. A conventional belt used to hold FHR transducers in place may, for example, be used for this purpose.

In some embodiments, the lower surface of the body has a flat perimeter region surrounding the protruding portion.

In some embodiments, the body of the device has a generally planar upper surface.

In some embodiments, the height of the protruding portion is between 15 mm and 35 mm.

In some embodiments, the lower surface of the body includes one or more engagement formations adapted to engage a cooperating engagement formation on the upper surface of the transducer.

In some embodiments, the engagement formation on the lower surface of the device body is an opening and the cooperating formation on the transducer is a button.

In some embodiments, the upper surface of the device body is configured to engage a belt that can be used to secure the device on the pregnant women's abdomen.

In some embodiments, a channel is formed on the upper surface of the device body to receive the belt.

In some embodiments, the device is formed from a medical grade polymer.

In some embodiments, the device is formed as a unitary moulded component.

In a second aspect, the invention provides an apparatus for securing a fetal heart rate transducer to the abdomen of a pregnant women, the system comprising:
a device according to the first aspect above; and
a fetal heart rate transducer, wherein in use a lower surface of the device is engaged with an upper surface of the transducer to push the transducer against the pregnant women's abdomen.

In some embodiments, the apparatus also comprises a belt adapted extend around the torso of the pregnant women, over the upper surface of the device, to apply pressure to the device to push it against the upper surface of the transducer.

The features of the device of the first aspect set forth above are equally applicable to the apparatus of this second aspect.

A third aspect of the invention provides a method for monitoring fetal heart rate, the method comprising:
placing a fetal heart rate transducer against a pregnant women's abdomen;
using a device having a lower surface with a protruding portion to apply pressure to a selected area on the upper surface of the transducer; and
using the transducer to detect a fetal heart rate signal whilst pressure is being applied to it by the device.

Preferably the method is carried out using a device according to the first aspect above.

The terms “upper” and “lower” and the like are used herein to define relative positions of elements or portions of elements and do not limit the device to any specific orientation in space. Generally, unless the context requires otherwise, the term “lower” is used to refer to a surface or other feature intended to face towards a pregnant women's abdomen in use and the term “upper” is used to refer to a surface or other feature intended to face away from a pregnant women's abdomen in use.

The skilled person will appreciate that the features described and defined in connection with the aspects of the invention and the embodiments thereof may be combined in any combination, regardless of whether the specific combination is expressly mentioned herein. Thus, all such combinations are considered to be made available to the skilled person.

### BRIEF DESCRIPTION OF THE DRAWINGS

FIG. 1 shows a perspective view of the lower surface of a device in accordance with an embodiment of the present invention;

FIG. 2 shows a perspective view of the upper surface of the device of FIG. 1;

FIG. 3 shows a plan view from below of the device of FIG. 1;

FIG. 4 shows a side view of the device of FIG. 1; and

FIGS. 5, 6 and 7 show examples of the device in situ on an abdomen being used to hold a FHR transducer in position.

### DETAILED DESCRIPTION OF THE EMBODIMENTS

An embodiment is described below by way of example with reference to the accompanying drawings.

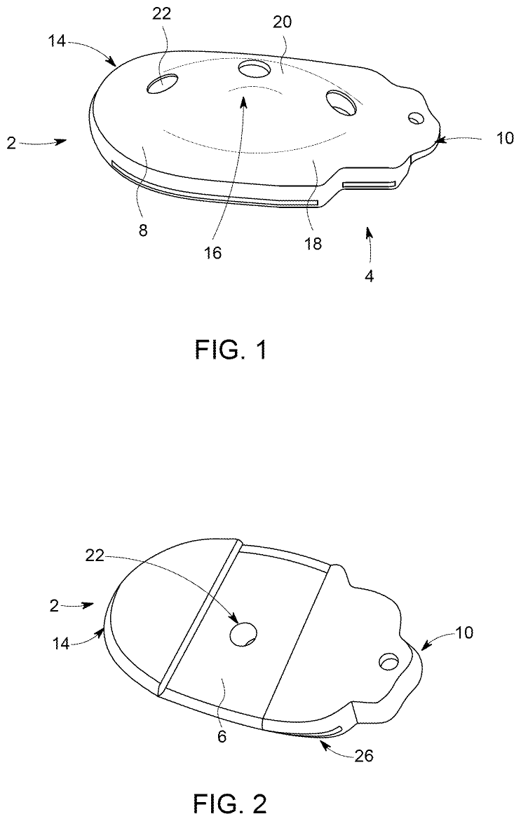

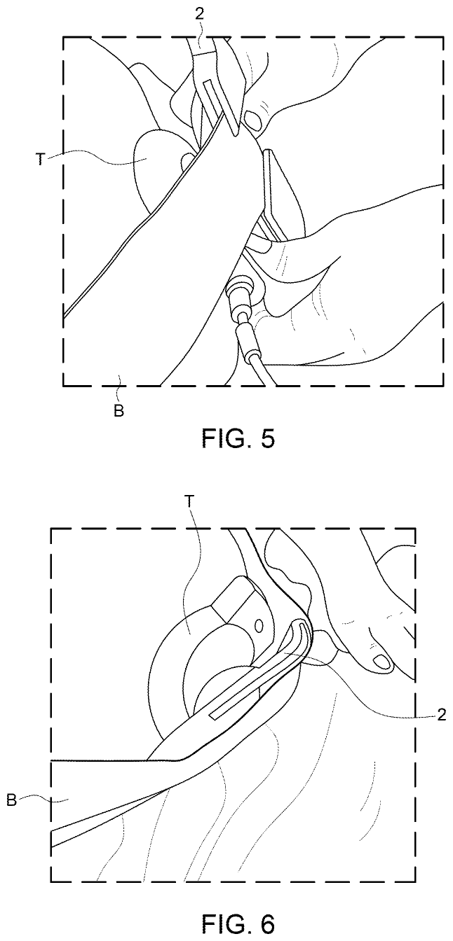

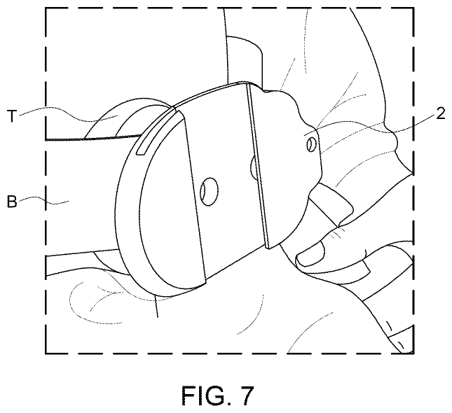

FIGS. 1 to 4 show an example of a device 2 that can be used, as shown in FIGS. 5 to 7, to hold an FHR transducer (T) in place against a pregnant women's abdomen either with (FIGS. 5 and 6) or without (FIG. 7) the use of a belt (B).

In this example, the device 2 is formed as a single unitary element. The device may be moulded from a medical grade polymer, for example a medical grade silicone. The body of the device is preferably flexible.

The body 4 of the device is generally oval in shape with a generally planar upper surface 6 and a non-planar lower surface 8. In this example, a front end 10 of the device is shaped (in plan view) with multiple steps 12 to allow it to be more easily gripped by a user (e.g. midwife) as they initially position the device 2. A rear end 14 of the body is generally semi-circular in shape (in plan view). Preferred examples of the device 2 have a shape designed to mimic the palm of a hand (and may be formed using a mould that has been shaped using a real person's hand).

As best seen in FIGS. 1 and 4, the lower surface 8 (i.e. the surface intended to face the woman's abdomen in use) has a central portion 16 that protrudes beyond a flat perimeter portion 18 of the lower surface 8.

The body 4, especially the protruding central portion 16, is shaped and sized so that the nose 20 of the bulge created by the protruding portion 16 (i.e. that part of the protruding portion that protrudes furthest from the flat perimeter portion) is smaller in surface area than the upper surface of the transducer (T) it is intended to be used with.

This configuration means that, in use, the central bulge 16 can be pushed against a selected area on the upper surface of the transducer (T) to apply pressure to that area, mimicking the manner in which a midwife would apply uneven pressure to the transducer to angle it relative to the abdomen to obtain a good FHR signal.

The nose 20 of the bulge can, for example, be placed centrally on the upper surface of the transducer (T) to apply an even (straight up and down) pressure to the transducer or more towards an edge of the transducer's upper surface to apply a pressure that tilts the transducer (T) towards that edge.

In the illustrated example, the height of the bulge (i.e. the distance it protrudes above the perimeter portion of the lower surface of the body) is about 25 mm. In other examples it may be more or less than 25 mm, for example in the range 15 mm to 35 mm.

The height of the bulge, as well as being a factor in the ability to use the device to apply pressure to the transducer at different angles, can also contribute to the weight of the device body. Specifically, if the bulge is formed as a solid portion of the body, a larger bulge will add more weight to the device. This in turn means the device is able to, itself, apply greater pressure to the transducer.

In the illustrated example, the length of the device body 4 is about 150 mm and the width is about 75 mm. Other dimensions are possible but typically the length will be in the range 100 mm to 200 mm and the width in the range 50 mm to 100 mm.

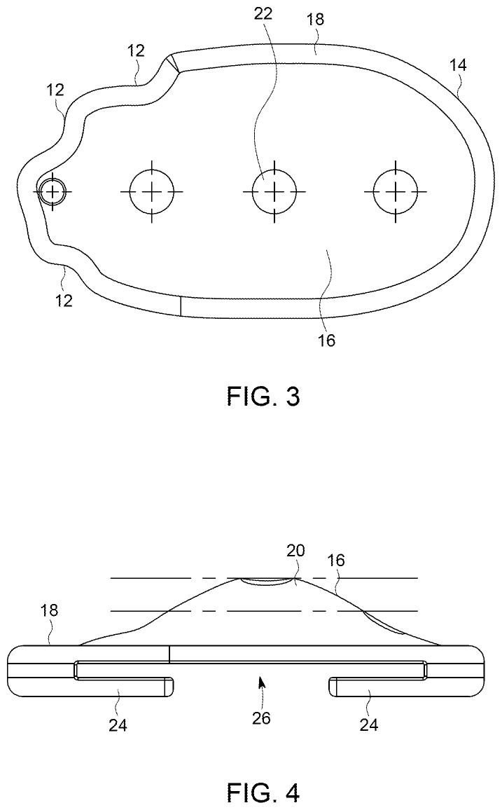

As seen best in FIGS. 1 and 3, the lower surface 8 of the device body 4 is formed with a row of three circular openings 22. These are configured to engage with a ‘button’ found on the upper surface of many conventional FHR transducers. These ‘buttons’ normally serve as attachment points for the belts that are traditionally used to hold the transducer in place.

In the illustrated example there are 3 of these circular openings 22, offering three different locations on the lower surface 8 of the body 4 that can be engaged with a ‘button’ on the transducer (T), to provide corresponding different orientations of the device relative to the transducer (T).

In the illustrated example, the device is adapted to be secured in place, against the upper surface of the transducer (T) using a belt (B). To help hold the belt (B) in place, the upper surface 6 of the device body 4 includes two flanges 24 that extend over end portions of the upper surface 6 (as best seen in FIG. 4) to form a channel 26 within which a belt (B) can be received. The flanges 24 are preferably flexible so they can be flexed away from the upper surface 6 of the device body 4, opening the channel mouth wider to more easily receive the belt (B).

In the illustrated example, the flanges 24 extend in from each end by about 25 mm, leaving a channel opening of about 35 mm.

The device 2 will typically be provided in a sterile condition in sterile packaging, as is conventional for medical equipment. The device 2 may be a single use device, intended to be disposed of once used, or it may be a multiple use device 2, intended to be sterilized between uses. Alternatively the device 2 may be self-sterilising.

In use, the device 2 is positioned on top of an already positioned FHR transducer (T) and can then be held in place with a belt (B), as shown for example in FIG. 5. The central bulge 16 on the lower surface 8 of the device body 4 is located on the surface of the transducer in the area where it is necessary to apply pressure to obtain a good FHR signal.

In late phases of labour, the device 2 can be used laterally, as shown in FIG. 6, to apply pressure on the transducer (T) around the symphysis pubis.

It is also possible to use the device 2 without a belt, as shown in FIG. 7, in cases where the weight of the device 2 itself is able to apply sufficient pressure to the transducer (T) to obtain a good FHR signal. In this case, the device 2 is mounted on the transducer (T) by engagement of one of the openings 22 with a button on the upper surface of the transducer (T).

The skilled person will understand that various modifications and additions can be made to the examples described above without departing from the spirit and scope of the present invention.

1. A device for positioning a fetal heart rate transducer against a pregnant women's abdomen, the device comprising:
a body having a lower surface and an upper surface opposite the lower surface;
wherein the lower surface is non-planar and comprises a protruding portion intended to engage an upper surface of the transducer in use, and configured such that the protruding portion can be positioned to apply a force to a selected area on the upper surface of the transducer.

2. A device according to claim 1, wherein the lower surface of the body has a flat perimeter region surrounding the protruding portion.

3. A device according to claim 1 wherein the body of the device has a generally planar upper surface.

4. A device according to claim 1, wherein the height of the protruding portion is between 15 mm and 35 mm.

5. A device according to claim 1, wherein the lower surface of the body includes one or more engagement formations adapted to engage a cooperating engagement formation on the upper surface of the transducer.

6. A device according to claim 5, wherein the engagement formation on the lower surface of the device body is an opening and the cooperating formation on the transducer is a button.

7. A device according to claim 1, wherein the upper surface of the device body is configured to engage a belt that can be used to secure the device on the pregnant women's abdomen.

8. A device according to claim 7, wherein a channel is formed on the upper surface of the device body to receive the belt.

9. A device according to claim 1, formed from a medical grade polymer.

10. A device according to claim 1, formed as a unitary moulded component.

11. An apparatus for securing a fetal heart rate transducer to the abdomen of a pregnant women, the system comprising:
a device according to claim 1; and
a fetal heart rate transducer, wherein in use a lower surface of the device is engaged with an upper surface of the transducer to push the transducer against the pregnant women's abdomen.

12. An apparatus according to claim 11, wherein an outer end of the protruding portion on the body of the device has a smaller surface area than the area of the upper surface of the transducer, whereby the device can be positioned to apply pressure to a selected area on the upper surface of the transducer.

13. An apparatus according to claim 11, further comprising a belt adapted extend around the torso of the pregnant women, over the upper surface of the device, to apply pressure to the device to push it against the upper surface of the transducer.

14. Use of a device according to claim 1, to hold a fetal heart rate transducer against a pregnant women's abdomen whilst recording a fetal heart rate signal.

15. A method for monitoring fetal heart rate, the method comprising:
placing a fetal heart rate transducer against a pregnant women's abdomen;
using a device having a lower surface with a protruding portion to apply pressure to a selected area on the upper surface of the transducer; and
using the transducer to detect a fetal heart rate signal whilst pressure is being applied to it by the device.

16. The method according to claim 15, wherein the device is the device according to claim 1.
